(19) United States
(12) Patent Application Publication
(10) Pub. No.: US 2023/0158256 A1
(43) Pub. Date: May 25, 2023

(54) INHALER, ASSEMBLY AND METHOD FOR INHALING INHALATION MEDIUM ENRICHED WITH ACTIVE INGREDIENTS AND/OR AROMATIC SUBSTANCES, AND METHOD FOR CONTROLLING AN INHALER

(71) Applicant: Körber Technologies GMBH, Hamburg (DE)

(72) Inventors: Christian Hanneken, Hamburg (DE); Michael Joseph Capone, Hamburg (DE)

(21) Appl. No.: 17/920,253

(22) PCT Filed: Apr. 12, 2021
(86) PCT No.: PCT/EP2021/059391

§ 371 Date: Oct. 20, 2022

(30) Foreign Application Priority Data
Apr. 23, 2020 (DE) 102020111034.0

(51) Int. Cl. A61M 11/04

(52) U.S. Cl. CPC A61M 11/042; A61M 2205/3569; A61M 2205/3584; A61M 2205/609

(57) ABSTRACT

An inhaler configured and adapted for inhaling an inhalation medium, enriched with active ingredients and/or flavouring substances, includes a cartridge carrier, a storage tank which contains the inhalation medium, a mouthpiece which is associated with the cartridge carrier, and an actuating mechanism for releasing the inhalation medium out of the storage tank in the direction of the mouthpiece. The inhaler has at least one sensor associated with the mouthpiece. The sensor is configured and adapted to detect characteristics of lips of the person using the inhaler and to provide a data signal formed from the characteristics. The sensor is connected to an electronic control unit associated with the inhaler, to which the actuating mechanism is also connected, in such a manner that the inhalation medium is released on the basis of the data signal. A corresponding arrangement and a corresponding method are disclosed.

### CROSS-REFERENCE TO RELATED APPLICATIONS

This application is the U.S. National Stage of PCT/EP2021/059391 filed on Apr. 12, 2021, which claims priority to German Patent Application 102020111034.0 filed on Apr. 23, 2020, the entire content of both are incorporated herein by reference in their entirety.

### FIELD OF THE INVENTION

The invention relates to an inhaler which is configured and adapted for inhaling an inhalation medium enriched with active ingredients and/or flavouring substances, comprising a cartridge carrier, a storage tank which contains the inhalation medium, a mouthpiece which is associated with the cartridge carrier, and an actuating mechanism for releasing the inhalation medium out of the storage tank in the direction of the mouthpiece.

The invention further relates to an arrangement which is configured and adapted for inhaling inhalation medium enriched with active ingredients and/or flavouring substances.

The invention is further concerned with a method for inhaling inhalation medium enriched with active ingredients and/or flavouring substances by means of an inhaler having a mouthpiece, comprising the following steps: Sucking at the mouthpiece of the inhaler by a user, and simultaneous actuation of an actuating mechanism of the inhaler, as a result of which the inhaler is activated for inhaling the inhalation medium.

The invention also relates to a method for controlling an inhaler.

### BACKGROUND OF THE INVENTION

Such inhalers and arrangements are used predominantly in the luxury goods/stimulants industry, here in particular in connection with an electronic cigarette (referred to in the following as an e-cigarette), as well as in the medical field to enable the inhalation of tobacco products and/or medical products, which are preferably present as a fluid inhalation medium, as an aerosol, in vapour form and/or as a mist. The user consumes the tobacco products and/or medical products using an inhaler which can be based, for example, on the principle of atomisation, nebulisation or vaporisation for providing the inhalation medium in a manner suitable for consumption or inhalation. When consuming, the user usually sucks on a mouthpiece of the inhaler. To do this, the person encloses at least parts of the mouthpiece with their lips and sucks, inhaling the inhalation medium, e.g. an aerosol. The drawing/sucking can be actively supported, e.g. by pumps or other actively driven and/or controlled means, or it can be replaced completely. Sucking alone or sucking in conjunction with additional manually or automatically, namely preferably electronically, controlled actuation of the actuating mechanism activates it such that an inhalation medium is released and can be inhaled.

The inhalation media frequently contain components, substances, active ingredients, additives or the like, e.g. nicotine, medical and/or therapeutic agents, that have restrictions on the authorisation to take them. The restrictions may be statutory in nature, e.g. the minimum age of the user for the consumption of nicotine, or based on physician recommendation/prescription for a particular person, such as available prescription-only medications. However, using e-cigarettes as an example, it is currently possible, for example, for an authorised person to purchase an e-cigarette and to pass it on to an unauthorised person for use. Using medical products as an example, there is a risk that a medicine, e.g. an inhaler with an asthma spray, that has been prescribed to a patient may end up in the hands of unauthorised persons, in particular children, who may suffer adverse health effects as a result of taking it. It is also conceivable that strong painkillers, prescribed to a patient for simplified administration by inhalation, could be used without authorisation by another person who is, for example, addicted to painkillers.

In many cases of use, it is therefore desirable or even necessary to verify the authorisation of the person using the inhaler in each case during each use, i.e. while the user is sucking on the inhaler, in order to effectively prevent misuse when purchasing the inhaler or to effectively prevent the purchase of the active ingredients and/or flavouring substances administered by it, but also in particular, during each use, to effectively prevent an unauthorised person from using it or them. To date, it is only possible to check that a person is authorised to obtain or purchase the inhaler when the inhaler, the active ingredients that can be vaporised by it, and the arrangements that are configured and adapted for inhalation are first dispensed or first acquired. To this end, the stores, shops, pharmacies or even online suppliers require a verification, for example by presenting a valid ID card, passport, prescription or the like, in order to verify that a person is authorised, e.g. their age for using nicotine and/or other active ingredients and flavouring substances for which a legal age restriction applies, or a valid prescription to take a medicine. Verification can be performed analogously, e.g. by the seller, or digitally, e.g. using a generally known authentication by smartphone app. After the inhaler has been purchased or handed over to an authorised person, the inhaler can actually be used without restriction. In particular, its use is possible regardless of the actual authorisation of the person using the inhaler. This can result in misuse by unauthorised persons. Existing security measures can also be circumvented by so-called straw purchases, i.e. targeted purchase by an authorised person for an unauthorised person. Particularly in the case of online verification by providing, e.g. age details, a credit card as proof or the like, the proof of authorisation can easily be circumvented by providing false information so that even the purchase can be abused easily. This means that the purchase alone but even more so the further use of an inhaler and an arrangement by persons who should not actually be allowed to use the inhalation medium for legal reasons (e.g. age restriction) and/or for health reasons (e.g. wrong medication) are basically possible without restriction.

On the one hand, the resulting disadvantages of known inhalers and arrangements with such inhalers consist, as mentioned, in the fact that verification can easily be circumvented even when purchasing an inhaler filled with inhalation medium. On the other hand, no known inhaler and no arrangement comprising an inhaler protects against misuse. In other words, the problem is that the known inhalers and arrangements, even if purchased legitimately, can be misused by someone who is not authorised.

The object of the invention is thus to create an inhaler that enables use exclusively for authorised/entitled persons. The object further consists of proposing a corresponding arrangement. A further object consists of proposing a corresponding method.

### SUMMARY OF THE INVENTION

This object is achieved by an inhaler of the type referred to hereinbefore in that at least one sensor is associated with the mouthpiece, which sensor is configured and adapted to detect characteristics of lips of the person using the inhaler and provide a data signal formed from the characteristics, the sensor being connected to an electronic control unit associated with the inhaler to which the actuating mechanism is also connected, in such a manner that the inhalation medium is released on the basis of the data signal.

It is possible with the embodiment according to the invention to verify the authorisation of the person using the device, i.e. the user, both during initial use and during each subsequent use, i.e. in particular during each individual sucking action. The invention makes it possible to allow or prevent the inhaler from being used depending on the person using it. The fact that the sensor is arranged on the mouthpiece and is enclosed by the lips of the user ensures, for example, that a lip wrinkle pattern or regions thereof are inevitably detected as a characteristic of the user during each use.

The print of the lips of a person is unique and, among other things, differs from a lip print of another person in the shape of the pattern of the lip grooves, i.e. in depth, width, length, arrangement and the course of the grooves in the outer skin layer(s) of the lip of a person. The specialised field dealing with this topic is called cheiloscopy. Due to the uniqueness of the lip print and its unambiguous assignability to a person, the lip print is ideally suited for identifying people. A minimal number of features of a shape of the pattern of lip grooves of a person, which is suitable for distinguishing the person from another person, is also referred to as characteristics of lips in the context of the present invention.

The lip wrinkle pattern or the data signal formed therefrom can be stored in the inhaler, an external device, such as a smartphone or a tablet PC, or in (cloud) storage. Knowing the respective lip wrinkle pattern or characteristics and the data signal formed therefrom, the actuating mechanism, which ultimately ensures whether or not the inhalation medium is released for inhalation by the user, can be controlled by means of the electronic control unit. Misuse is virtually impossible, as the mouthpiece must be placed in the mouth and thus between the lips of the user during each individual sucking action to verify authorisation. Once the mouthpiece is held between the lips of the user authorised to use it, the mouthpiece can no longer be held in the mouth or between the lips of a further user. This makes it virtually impossible to separate the authorised user from another, non-authorised user of the inhaler with the inhaler according to the invention. In other words, to use the inhaler, the user who inhales the inhalation medium must be identical to the authorised user.

Recording and identifying characteristics of the lips can be done in different ways. The recording of characteristics can be implemented, for example, with sensors. To record characteristics of the lips, sensors acquire a spatially and preferably height-resolved image of the lips, i.e. a map of the lip print. The data signal generated by the sensors during recording can be abstracted to a biometric signature using a suitable algorithm while retaining distinctive features in the lip print. This biometric signature forms the basis for the further work of the electronic control device in the inhaler. Distinctive features can be, for example, the spatial arrangement of whorls of lip grooves with respect to each other and their degree of curvature, the shape of lip grooves or the spatial arrangement of branching points where one lip groove, for example, merges into two lip grooves.

For example, a single print of the lips can be recorded as a data signal for the initial recording of the characteristics of the lips of the authorised user. Optionally, different regions of the lips can be recorded once or repeatedly by moving the mouthpiece between the lips. In this case, it is conceivable that by taking several images of regions of the lips, a map of the lip print is created from all the regions recorded and thus an image of the entire relevant portion of the lips is created. “Relevant portion of the lips” means the region of the lips of the user that usually touches the sensors associated with the mouthpiece when using the mouthpiece of the inhaler. This map can then be abstracted in turn to a biometric signature.

Furthermore, it is conceivable that the characteristics of the lips of the authorised user are extracted from a photo of the user. For example, the photo can be taken as part of a verification process with a mobile device, e.g. a smartphone or a tablet PC, as is known in prior art, for example, when setting up an account or a contract. Such a photo, for example, can be taken with the aid of a smartphone. In this case, at the same time as verifying that the user is authorised, inhaler-independent characteristics of the lips of the previously or simultaneously verified user can be captured or created by comparing the photo taken of the user with the photo of an official ID card of the same user. These characteristics can then be backed up to cloud storage. It is then conceivable that the characteristics are uploaded to and stored in a storage device of the inhaler or the cartridge carrier when the inhaler or the cartridge carrier is put into operation for the first time, so that verification according to the invention by the control device of the inhaler can be carried out without a connection to the cloud storage.

Detection of the lip wrinkle pattern or parts thereof by two or more sensors is possible and is described in greater detail below. The information/data detected by the or each sensor can be processed individually or combined with each other into a plurality of data signals or a common data signal.

Advantageously, the inhaler is characterised in that the actuating mechanism comprises a locking mechanism which is configured and adapted to enable and prevent release of the inhalation medium by means of the electronic control unit based on the data signal. The controllable locking mechanism ensures individual and verified release of the inhalation medium.

A preferred further development of the inhaler is characterised in that the locking mechanism is configured to be mechanical and/or electronic and/or electromechanical and/or electromagnetic and/or computer-implemented or a combination of the aforementioned. The locking mechanism as a mechanical component may comprise, e.g. a latch, a catch or the like. The mechanically configured locking mechanism can be moved from a closed position, i.e. a position in which release of the inhalation medium is not possible, to a release position, i.e. a position in which release of the inhalation medium is possible, and vice versa. It is also conceivable that the mechanically configured locking mechanism is formed by the relative displacement of two parts of the inhaler against each other, e.g. against a restoring spring force, and thereby the two parts can be brought from the closed position into the release position. Furthermore, it is conceivable that the mechanically configured locking mechanism is formed by pretensioning one part of the inhaler with respect to another part of the inhaler. In this case, the pretensioning can be achieved, for example, by a rotating and/or pulling movement of the part of the inhaler with respect to the other part of the inhaler and against a spring force due to a manual execution by the user. The rotating and/or pulling movement can apply a spring force to a part of the inhaler in a closed position. By actuating a mechanical trigger, e.g. by releasing a locking device due to manual operation by the user, the spring force can move the spring-loaded part of the inhaler from the closed position to the release position. In other embodiments, electronic or electromechanical activation of the mechanical components is possible.

For example, the aforementioned mechanical trigger can be formed by or actuated by one element or a combination of a plurality of the elements of the following list: electro-mechanical component, electromagnetic actuating drive, electric motor, servomotor, stepper motor, electromechanically/electromagnetically-operated piston, electro-mechanically/electromagnetically-operated diaphragm, solenoid coil that changes its position when current flows through the solenoid coil. The locking mechanism, however, can also be configured purely electrically/electronically overall, in that the release and locking takes place on the basis of e.g. data signals or the like. However, it is also conceivable that the locking mechanism is computer-implemented and executed on a microcontroller. For this purpose, a microcontroller, which, for example, is part of the control unit, can have and execute an algorithm stored in a data storage device. The algorithm can be configured and adapted in such a manner that it converts the data signal measured by the sensor into a data set that allows this data set to be compared with a previously known data set. Furthermore, the algorithm can be adapted to compare the data set with the previously known data set. The algorithm can then be configured to actuate an output of the microcontroller if the data set matches the previously known data set, as a result of which, for example, an electronic release device can be actuated and the inhalation medium released.

An expedient embodiment is characterised in that the inhaler comprises an element that is configured and adapted to convert the data signal formed by the or each sensor into a biometric signature, the biometric signature forming an electronic lock as the locking mechanism for the actuating mechanism. If there is an existing match with an initially detected and stored biometric signature, as described above for example, the inhalation medium can be released. Without a match with an initially detected and stored biometric signature, the actuating mechanism remains locked. Expressed differently, if there is no match, no inhalation medium is released.

A preferred embodiment is characterised in that the sensor is a skin print sensor or the sensor comprises a skin print sensor. A skin print sensor is a sensor that is adapted to record a skin print, such as a lip wrinkle pattern. In this case, the skin print sensor measures a sample of a lip wrinkle pattern and converts it into a data signal. Recording of the skin print by the skin print sensor can be controlled by an algorithm, for example a readout and conversion algorithm, in the skin print sensor or in the electronic control unit of the inhaler, e.g. a microcontroller of the inhaler. The skin print sensor, for example, can record a spatially-resolved image, i.e. a map of the lip structure of a specific region of the lips. The skin print sensor can preferably be an optical, capacitive, optical-capacitive, pressure-sensitive, thermal or ultrasound-based sensor.

A preferred embodiment is characterised in that the mouthpiece has a respective sensor arranged on a side of the mouthpiece substantially facing the upper lip and on a side of the mouthpiece substantially facing the lower lip when the mouthpiece is received as intended between the lips of a user. Alternatively or additionally, it is also conceivable that instead of the at least two sensors described above, the mouthpiece has a circumferential sensor that performs the same function of simultaneously measuring the lip print of a region of the upper lip and a region of the lower lip. The combination of at least two sensors or a circumferential sensor and the preferably simultaneous measurement of the lip wrinkle pattern of a region of the upper lip and a region of the lower lip by a separate sensor in each case ensures that both lips of the verifying person are unambiguously the lips of the authorised user when using the inhaler. This significantly reinforces the protection against unauthorised use of the inhaler.

Advantageously, the electronic control unit is configured as a programmable logic control unit which is configured and adapted to store the biometric signature and to read out the biometric signature from the storage device. This embodiment ensures easy and quick verification for a kind of on-use authorisation check of the person using it, i.e. an authorisation check during use of the inhaler. As mentioned, storage may be in the inhaler itself or in an external device or (cloud) storage.

A preferred embodiment is characterised in that the inhaler comprises an element which is configured and adapted for digital verification of the identity and/or age of the person using the inhaler. This further increases security during verification and makes misuse more difficult. From the digital proof of identity and/or age, on the one hand, and the lip wrinkle pattern, on the other hand, a data set can be formed which is stored to enable a comparison with the current lip wrinkle pattern at each subsequent use. The possibilities of digital proof of identity and/or age and matching for verification are diverse. For example, the person using it can provide a digital copy of an ID card, passport, driving licence, or the like, and take a so-called selfie of themselves with the mouthpiece between their lips. By means of a digital query, e.g. on government/public databases or the like, the validity of the ID card, passport, driving licence, etc., the age determination or the like can be checked and the selfie can be matched with the photo of the ID card, passport, driving licence, etc.

A particularly advantageous further development is characterised in that, for initial verification, the programmable logic control unit is configured and adapted to form a data set formed from the biometric signature and a digital proof of identity and/or age of the user and to store and read out the data set. The inhaler according to the invention makes it possible, before using the inhaler for the first time, to establish a connection between the identity of the person using the inhaler, e.g. based on a photo taken when it is used for the first time and a comparison with the photo of a national ID card, and a lip wrinkle pattern of the user as a characteristic. For this purpose, the identity and/or the age of the user is/are determined by the technical means mentioned and the lip wrinkle pattern of the user is detected by means of the sensor and converted into the biometric signature. If the person using the device is authorised, the data set formed from the authorisation and the signature is stored, it being possible for the storage to be in the inhaler, in an external device, in (cloud) storage or the like.

For re-verification, the programmable logic control unit is expediently configured and adapted to compare the stored data set with a data signal actively formed from the characteristics during each individual use. During the first and each subsequent use of the same inhaler, the embodiment according to the invention can be used to compare the lip wrinkle pattern detected in the person currently using the inhaler, as a characteristic or the biometric signature formed from it, with the lip wrinkle pattern initially detected and stored or the biometric signature formed therefrom. If the biometric signatures match or are similar to a defined minimum degree, the inhaler is enabled for use. Expressed differently, when a matching result is obtained, the locking mechanism of the actuating mechanism is unlocked. If there is no match, the locking mechanism remains locked so that the actuating mechanism cannot be triggered. Ultimately, the invention enables on-use or online verification of the user in each case so that misuse is ruled out. Thus, the invention enables effective and safe handling of the inhaler exclusively by authorised persons, in that verification of the person and use by the person are quasi synchronous. Expressed differently, verification is checked during each use.

An advantageous embodiment is characterised in that the inhaler comprises an element which is configured and adapted to establish a wireless connection to a network-enabled terminal. This significantly simplifies handling of the inhaler. The aforementioned benefits are further supported by this development. It is particularly advantageous that the user can use the terminal device, for example a smartphone or a tablet, to set up an app or an account in order to digitally verify their identity and/or their age. The terminal device, however, can also be a terminal at a retailer's premises, a store, a pharmacy, a doctor's surgery or the like.

Particularly preferred is an inhaler which is characterised in that a vaporiser cartridge is associated with the cartridge carrier, an energy source being associated with a unit formed from the cartridge carrier and vaporiser cartridge, and in that the vaporiser cartridge has a hollow body with a continuous flow channel, the storage tank being configured to receive a liquid inhalation medium and having at least one access opening to the flow channel and a vaporiser unit, which extends over the entire access opening, being arranged in the region of each access opening. In this embodiment, the vaporiser unit, due to heating up the fluid, ensures that aerosols can be formed and inhaled due to the air flow created by sucking. Optionally, the aerosols can also be formed by ultrasound devices. However, it is also conceivable that aerosols are formed by releasing the inhalation medium through a nozzle of a pressurised container which is housed in or on the inhaler. Alternatively to the pressurised container, the inhaler can also comprise an apparatus which generates a pressure that is suitable for producing aerosols through a nozzle.

The vaporiser unit preferably has a wick member and a heating member, the vaporiser unit comprising electrical contacts for electrically contacting the energy source. By sucking on the mouthpiece, the authorisation of the person using the inhaler is checked via the sensor and, if the user is authorised, the heating pulse for vaporising the liquid is initiated. Thus, the biometric signature formed from the lip wrinkle pattern forms the digital/electronic lock for the inhaler. Only when the lock is opened, namely when authorisation is proven by sucking on the mouthpiece, do the aerosols flow through the flow channel and can be inhaled by the person using the inhaler. If there is no authorisation, there is also no heating pulse for the vaporiser unit. In other words, the lock remains locked.

Particularly preferably, the vaporiser unit with the energy source forms the actuating mechanism, it being possible to control and/or regulate the actuating mechanism by means of the electronic control unit. The resulting advantages have already been explained above.

The object is also achieved by an arrangement which is characterised by an inhaler as disclosed herein, and a network-enabled terminal device, it being possible to bring the inhaler and the terminal device wirelessly into signal connection with each other. For example, the inhaler and the terminal device can communicate with each other via Bluetooth. Optionally, communication via a local or global network is also possible. Via the Bluetooth or network interface, the inhaler is linked in particular to the account set up on the terminal device. For this purpose, the terminal device preferably has a user-defined account which is linked to the inhaler.

Expediently, the inhaler and/or the terminal device can be wirelessly connected to a network. For example, the inhaler, as described above, is connected to the terminal device via a Bluetooth interface. The terminal device in turn is wirelessly connected to a network, preferably the Internet as a global network. Optionally, the inhaler can also communicate wirelessly, e.g. via radio connection, such as Bluetooth, RFID, NFC or ZigBee, directly or via intermediate network components, such as WLAN/WiFi via the network (e.g. Internet), e.g. with other terminal devices or further arbitrary network components, such as a server.

The object is further achieved by a method for inhaling of the type referred to hereinbefore which is characterised by the following steps: A connection between the identity and/or age of the user and characteristics of the lips of the user is established and stored before sucking for the first time and, preferably during each suck, a comparison of the characteristics of the lips of the person currently using the inhaler is made with the characteristics initially stored, in such a manner that activation of the actuating mechanism only takes place if the respective characteristics match to a defined minimum degree.

Advantageously, the initially detected and saved connection is stored as authorisation to use the inhaler.

A preferred development is characterised in that a lip wrinkle pattern of the user is detected by means of a sensor and converted into a biometric signature, the biometric signature being linked to an authorisation to use the inhaler and deactivating the actuating mechanism as an electronic lock until the authorised person sucks on the inhaler.

Particularly preferably, the method is carried out using an arrangement as disclosed herein.

The object is further achieved by a method for controlling of the type referred to hereinbefore. The method is a method for controlling an inhaler having a mouthpiece with a sensor, as a result of which the inhaler is activated for inhaling an inhalation medium, comprising the following steps: Recording of data, preferably characteristics of the lips, of a person currently using the inhaler by the sensor, comparing the recorded data of the current user with initially stored data, preferably initially stored data of the lips, and, if the initially stored data matches the recorded data of the current user to a defined minimum degree, enabling the actuating mechanism of the inhaler and preferably activating the actuating mechanism of the inhaler.

The advantages arising in connection with the arrangement and methods have already been described in detail in connection with the inhaler, which is why, to avoid repetition, reference is made to the preceding remarks which are achieved in a corresponding manner.

### BRIEF DESCRIPTION OF THE DRAWINGS

Further expedient and/or advantageous features and development of the inhaler and the arrangement as well as corresponding steps for the method emerge from the description. Particularly preferred embodiments of the inhaler as well as of the arrangement and the method are explained in greater detail with reference to the attached drawings.

FIGS. 1a and 1b are perspective views of different embodiments of inhalers according to the invention;

FIG. 2 is a schematic view of an arrangement, formed from an inhaler and a network-enabled terminal device, which is connected to a network;

FIG. 3 is a schematic and enlarged sectional diagram of the inhaler shown in FIG. 1b;

FIG. 4 is a schematic view of the inhalers according to FIGS. 1a and 1b with direct connection to a network; and

FIG. 5 is a schematic view of the inhalers according to FIGS. 1a and 1b with a connection to a terminal device via a Bluetooth interface.

### DETAILED DESCRIPTION OF THE INVENTION

The inhalers according to the invention shown in the drawing are configured, purely by way of example, optionally as an inhaler for medication based on the principle of atomisation or as a so-called e-cigarette based on the principle of vaporisation. Accordingly, the invention relates similarly to inhalers for enjoyment purposes and inhalers for medical and/or therapeutic purposes. Ultimately, the invention relates to all inhalers, i.e. means, which are configured and adapted for sucking in/breathing in/consuming or otherwise orally ingesting aerosols, liquid and/or vaporous media, for which a person using the inhaler must prove before using it for the first time that they are authorised to inhale the inhalation medium, and in each subsequent use process it is verified whether the person using the inhaler is the verified and authorised person.

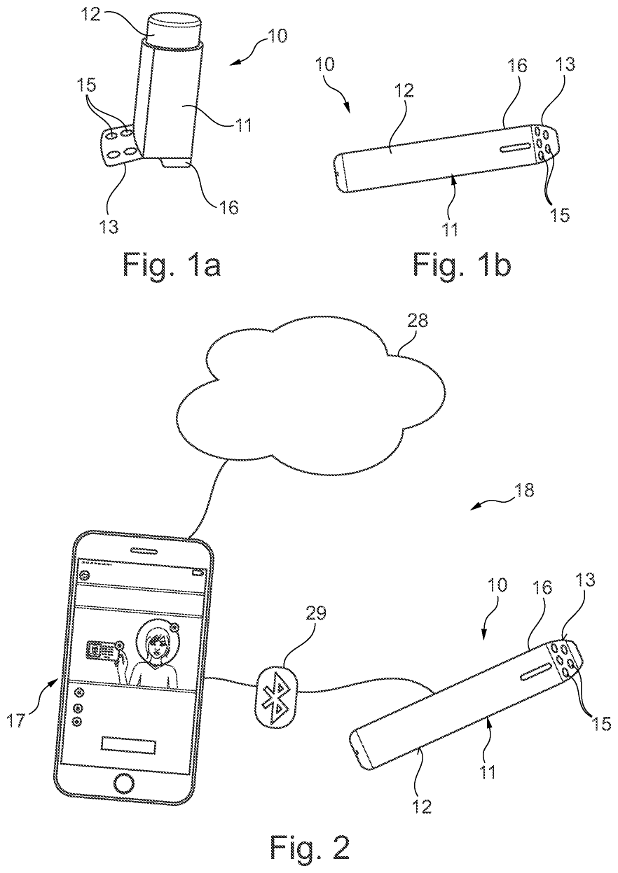

FIG. 1a shows an inhaler 10 which is configured and adapted for inhaling an inhalation medium enriched with active ingredients and/or flavouring substances for medical purposes. FIG. 1b shows an inhaler 10 which is configured and adapted for inhaling an inhalation medium enriched with active ingredients and/or flavouring substances for enjoyment purposes, i.e. a so-called e-cigarette.

Each inhaler 10 comprises a cartridge carrier 11, a storage tank 12 which contains the inhalation medium, a mouthpiece 13 which is associated with the cartridge carrier 11, and an actuating mechanism 14 for releasing the inhalation medium out of the storage tank 12 in the direction of the mouthpiece 13. Association of the mouthpiece 13 with the cartridge carrier 11 describes both mouthpieces 13 which are fixedly connected to the cartridge carrier 11 and mouthpieces 13 which are releasably attached to the cartridge carrier 11 in a replaceable manner.

The inhaler 10 can be based on different operating principles with respect to providing the inhalation medium in a manner suitable for consumption or inhalation. The inhaler 10 according to FIG. 1a is based on the principle of atomisation. The inhaler according to FIG. 1b is based on the principle of vaporisation. However, other operating principles, such as ultrasound or the like, can also be used as long at they convert a solid, liquid or gaseous inhalation medium into a state suitable for inhalation.

The actuating mechanism 14 describes in the broadest sense everything (mechanical and/or electronic) which is configured and adapted, on the one hand, to prevent undesirable emptying of the inhalation medium from the storage tank and escape from the mouthpiece and, on the other hand, to ensure targeted release of the inhalation medium directly, namely without a change, e.g. by nebulisation, atomisation, vaporisation or the like, or indirectly, namely with a change, e.g. by nebulisation, atomisation, vaporisation or the like, from the storage tank 12 towards the mouthpiece 13. The storage tank 12 can be part of the cartridge carrier 11 or can be configured separately. It is crucial that there is a connection between the storage tank 12 and the mouthpiece 13 in such a manner that the inhalation medium can be ingested or inhaled by suction directly, e.g. as a liquid, or indirectly or in converted form, e.g. as an (atomised or vaporised) liquid to form an aerosol or the like.

The inhaler 10 is characterised according to the invention in that at least one sensor 15 is associated with the mouthpiece 13, which sensor is configured and adapted to detect characteristics of lips of the person using the inhaler 10 and provide a data signal formed from the characteristics, wherein the sensor 15 is connected to an electronic control unit 16 associated with the inhaler 10, and to which the actuating mechanism 14 is also connected, in such a manner that the inhalation medium is released on the basis of the data signal.

The sensor 15 can be, for example, a single ring sensor. It is also possible to have two or more than two sensors 15, preferably evenly distributed around the circumference of the mouthpiece 13. In the case of a flat mouthpiece 13 with two opposing surfaces, for example, at least one sensor 15 can be arranged in the region of each surface. However, other sensor configurations and sensor positions are also possible in the region of the mouthpiece 13.

When taken on their own and in combination with each other, the features and developments described below illustrate preferred embodiments. It is expressly pointed out that features which are summarised the description and/or the drawing or are described in a common embodiment can also further develop the inhaler 10 described above in a functionally independent manner.

The actuating mechanism 14 comprises a locking mechanism (not explicitly illustrated) which is configured and adapted to enable and prevent release of the inhalation medium by means of the electronic control unit 16, based on the data signal or even a plurality of data signals. This locking mechanism can be configured mechanically and/or electronically. The locking mechanism causes the actuating mechanism 14 to be operable, releasing the inhalation medium accordingly, when the locking mechanism is out of (mechanical and/or electrical/electronic) engagement, and to lock the actuating mechanism 14, retaining the inhalation medium accordingly, when the locking mechanism is in (mechanical and/or electrical/electronic) engagement.

The inhaler 10 optionally further comprises an element, e.g. a processor, a program module or the like, (also not explicitly illustrated) that is preferably part of the control unit 16 and is configured and adapted to convert the data signal formed by the or each sensor 15 into a biometric signature, the biometric signature forming an electronic lock as a locking mechanism for the actuating mechanism 14. Simply put, the signature individual to each person forms not only the lock for the actuating mechanism 14 but at the same time the key as well.

The electronic control unit 16 is preferably configured as a programmable logic control unit which is configured and adapted to store the biometric signature and to read out the biometric signature from the storage device. In other words, the control unit 16 manages both the lock and the key in that the initially saved signature, which is stored as an access authorisation, basically blocks the actuating mechanism 14, and each time the inhaler 10 is used, it compares the signature then recognised or detected with the stored access authorisation and only releases the actuating mechanism 14 in the event of a previously defined, sufficient match.

In individual embodiments the inhaler 10 comprises an element which is configured and adapted for digital verification of the identity and/or age of the person using the inhaler 10. The element can be part of the inhaler 10. Particularly preferably, however, the element is an external terminal device 17 as part of an arrangement 18 which is described below.

For initial verification, the electronic or programmable logic control unit 16 is configured and adapted to form a data set formed from the biometric signature and a digital proof of identity and/or age of the user, and to store and read out the data set. For re-verification, the electronic or programmable logic control unit 16 is additionally configured and adapted to compare the stored data set with a data signal currently formed from the characteristics during each individual use. In this way, when the inhaler 10 is used for the first time, said inhaler 10 is used in a simple manner to establish a unique, individual connection between the identity of the person using the inhaler, e.g. based on a photo taken before the inhaler is used for the first time and comparison with a photo of a nationally recognised and stored ID card, and the lip wrinkle pattern of the user. The biometric signature formed from the lip wrinkle pattern is stored together with the identity of the user (e.g. legal minimum age according to the ID card) and/or their permission to use the inhaler (e.g. according to the prescription) as a data set. During further use of the inhaler 10, the lip wrinkle pattern or the biometric signature formed therefrom of the person currently using the inhaler is compared with the biometric signature initially stored. If there is a match or a defined minimum degree of similarity, the inhaler 10 is activated for use. If there is no match or a lack of similarity, the inhaler 10 remains deactivated. This matching takes place anew with each individual sucking action.

The inhaler 10 preferably comprises an element which is configured and adapted to establish a wireless connection to a network-enabled terminal device 17. The element is preferably a Bluetooth interface. In further embodiments, other interfaces and/or data/signal transmission components can also be used to establish communication between the inhaler 10 and the terminal device 17. Smartphones, tablets or the like are preferably used as terminal devices 17.

In a particularly preferred embodiment, in which the inhaler 10 is configured as a so-called e-cigarette, a vaporiser cartridge 19 is associated with the cartridge carrier 11, an energy source 20 being associated with a unit formed by the cartridge carrier 11 and the vaporiser cartridge 19. The vaporiser cartridge 19 has a hollow body 21 with a continuous flow channel 22, the storage tank 12 being configured to receive a liquid inhalation medium and having at least one access opening 23 to the flow channel 22 and a vaporiser unit 24, which extends over the entire access opening 23, being arranged in the region of each access opening 23. The vaporiser unit 24 has a wick member 25 and a heating member 26, the vaporiser unit 24 comprising electrical contacts 27 for electrically contacting the energy source 20. The vaporiser unit 24 is configured to be liquid-permeable in such a manner that liquid can be conveyed at least initially by capillary action from the storage tank 12 through the vaporiser unit 24 towards the flow channel 22.

The hollow body 21 with its at least one flow channel 22, two or more flow channels 22 can also be provided, forms a suction channel/vent. The shape of the hollow body 21 can be almost arbitrary, as can the course of the flow channel 22. What is crucial is that an inlet side of each flow channel 22 is open to the environment, for example to allow air to be drawn in, and that an outlet side is open, for example, to allow a negative pressure to be created, in particular due to the sucking of a user. Open in this context means that the inlet side and the outlet side are permeable to air. In the region of the access opening 23 between storage tank 12 and flow channel 22, the vaporiser unit 24 forms a kind of liquid barrier which prevents liquid from flowing from the storage tank 12 directly and as liquid into the flow channel 22. Regardless of the shape and configuration of the storage tank 12, two or more storage tanks 12 may also be provided, and of the hollow body 21 and of the arrangement/positioning of storage tank 12 to hollow body 21, the vaporiser unit 24 ensures that liquid is necessarily guided out of the storage tank 12 towards the flow channel 22 and is discharged into the flow channel 22 as gas or vapour for the formation of aerosols, at the latest when it exits from the vaporiser unit 24.

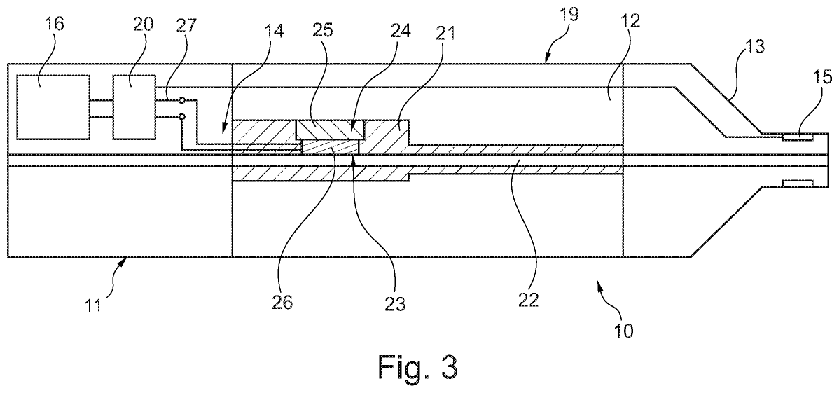

In the preceding example (e.g. according to FIGS. 1 and 3), the vaporiser unit 24 with the energy source 20 forms the actuating mechanism 14, it being possible to control and/or regulate the actuating mechanism 14 by means of the electronic control unit 16. When the control unit 16 activates the energy source 20, energy is supplied to the vaporiser unit 24 via the electrical contacts 27 which results in a vaporisation process. The initially detected and stored biometric signature forms the electronic lock as the locking mechanism for the actuating mechanism 14. If a verified authorisation is available during use, current signature corresponds to the initially detected and stored signature, the actuating mechanism 14 can be activated. If no verified authorisation is available, current signature does not correspond to the initially determined and stored signature, no activation of the actuating mechanism 14 can take place. In other examples (e.g. according to FIG. 1), the actuating mechanism 14 can be e.g. a spring element, a valve, a push-button or the like or a combination thereof, which can be released or locked e.g. by a latch, a catch or the like as a locking mechanism.

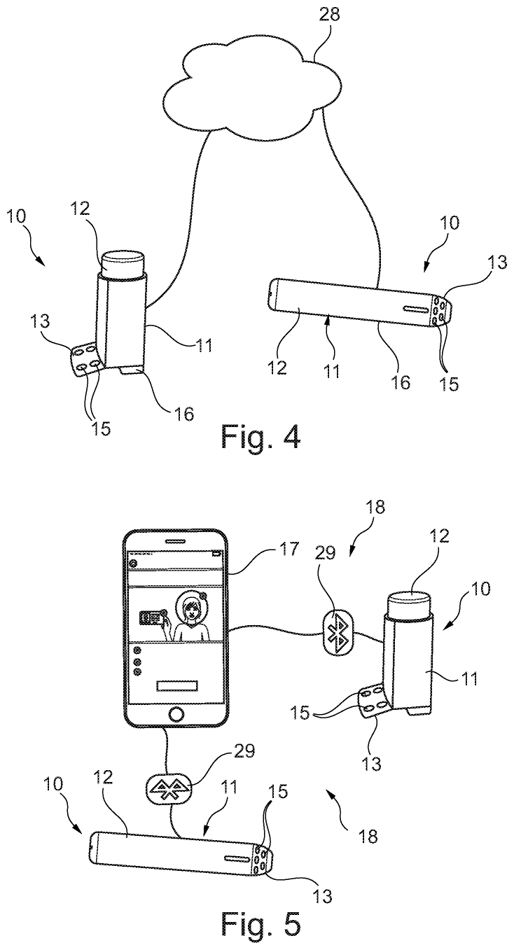

As can be seen from the foregoing, the inhaler 10 (see FIGS. 1a and 1b and 4) itself may include all components that ensure verified release or actuation of the actuating mechanism 14 each time it is used, i.e. sucking action by sucking action. Preferably, however, the inhaler 10 is part of an arrangement 18 which is configured and adapted for inhaling an inhalation medium enriched with active ingredients and/or flavouring substances. The arrangement 18 comprises an inhaler 10 as disclosed herein, and a network-enabled terminal device 17, it being possible to bring the inhaler 10 and the terminal device 17 wirelessly into signal connection with each other. Preferably, the inhaler 10 and/or the terminal device 17 can be wirelessly connected to a network 28. In the example according to FIG. 2, the inhaler 10 and the terminal device 17 are connected to each other via a Bluetooth interface 29. The terminal device 17 communicates with other terminal devices, such as servers or the like, via the network 28. In other variants, the inhalers 10 are connected directly to the network 28. However, other links and connections to local or global networks are also possible. The terminal device 17 preferably has a user-defined account which is linked to the inhaler 10. A so-called application, by means of which, for example, the identity and/or age verification can be carried out, is loaded onto the terminal device 17. The terminal device 17 with the application can also be used as a storage device for the data detected and formed, in particular the combination of biometric signature and identity and/or age information. However, the storage may also be associated with the network 28 as so-called cloud storage.

The method according to the invention is explained in greater detail below based on the drawing:

The method is used for inhaling inhalation medium enriched with active ingredients and/or flavouring substances. The inhalation medium may originally be solid (e.g. in the form of powder or the like), liquid or vaporous/gaseous. In this context, inhalation includes not only the consumption of converted inhalation media, i.e. for example, the inhalation of aerosols formed from solid and/or liquid media, which were formed, for example, by a vaporisation process or ultrasound process from a liquid supply or by atomisation from a supply of powder, but also the consumption of unconverted inhalation media, i.e. for example, the portionwise/dosewise dispensing of individual liquid portions/doses from a liquid supply.

To inhale, a user sucks on the mouthpiece 13 of the inhaler 10 and simultaneously activates an actuating mechanism 14 of the inhaler 10, as a result of which the inhaler 10 is activated for inhaling the inhalation medium. Activating the actuating mechanism 14 can be a separate step from sucking. However, sucking can simultaneously be the activation. By activating the actuating mechanism 14, the inhalation medium is released from a storage tank 12 towards the mouthpiece 13. In this case, the inhalation medium can reach the mouthpiece 13 unchanged/unconverted. Optionally, the inhalation medium is converted, namely, for example, atomised, vaporised, nebulised or otherwise altered, on the way from the storage tank 12 to the mouthpiece 13.

In an inhaler 10 according to FIG. 1a, the actuating mechanism 14 is activated, for example, by pressing on a storage tank 12 containing the inhalation medium, for example, by opening an atomising valve. By simultaneously sucking on the mouthpiece 13, the atomised inhalation medium can be inhaled as an aerosol. In an inhaler 10 according to FIG. 1b, the actuating mechanism 14 is already activated by the sucking itself, for example, by a sensor detecting the air flow and signalling a control unit 16 to activate an energy source 20 by means of which a vaporiser unit 24 initiates a vaporisation process in the inhaler 10. As a result, the liquid inhalation medium is vaporised and, when it enters a flow channel 22, is entrained by the sucking and converted into aerosols which are then inhaled.

According to the invention, a connection between the identity and/or age of the user and characteristics of the lips of the user is established and stored before sucking for the first time and, preferably during each suck, a comparison of the characteristics of the lips of the person currently using the inhaler is made with the characteristics initially stored, in such a manner that activation of the actuating mechanism 14 only takes place if the respective characteristics match to a defined minimum degree. The authorisation is verified and stored, so to speak, via the initial verification. With re-verification, the matching of characteristics is performed for each individual sucking action, such that activation of the inhaler 10 can only be initiated by the authorised person during use. For this purpose, the initially detected and saved connection is stored as authorisation to use the inhaler 10.

Specifically, a lip wrinkle pattern of the person using the inhaler is detected by a sensor 15 and converted into a biometric signature, the biometric signature being linked to an authorisation to use the inhaler and deactivating the actuating mechanism 14 as an electronic lock until the authorised person sucks on the inhaler 10. Checking the authorisation of the person using the inhaler can be done, for example, in that users take a photo of themselves together with a photo ID or the like and the mouthpiece 13 of the inhaler 10 in their mouth and have it verified by official/government sources, for example using databases or the like.

Particularly preferably, the method is carried out using an arrangement 18 as disclosed herein.

For the method, a communication environment can be determined or created for the inhaler 10 which makes it possible to communicate with at least one second device. This communication can take place via local or global networks. The data exchanged between inhaler 10 and terminal device 17, on the one hand, and between terminal device 17 and/or inhaler 10 via the network 28, on the other hand, can be sent and received unencrypted or encrypted using conventional methods. It is further possible to allow a plurality of authorised persons to use the inhaler 10.

1-21. (canceled)

22. An inhaler, configured and adapted for inhaling an inhalation medium enriched with active ingredients and/or flavouring substances, comprising:
a cartridge carrier;
a storage tank which contains the inhalation medium;
a mouthpiece associated with the cartridge carrier;
an actuating mechanism for releasing the inhalation medium out of the storage tank in a direction of the mouthpiece;
at least one sensor associated with the mouthpiece, the sensor being configured and adapted to detect characteristics of lips of a person using the inhaler and to provide a data signal formed from the characteristics; and
an electronic control unit, the sensor and the actuating mechanism being connected to the electronic control unit such that the inhalation medium is released on a basis of the data signal.

23. The inhaler according to claim 22, wherein the actuating mechanism comprises a locking mechanism configured and adapted to enable and prevent release of the inhalation medium by the electronic control unit based on the data signal.

24. The inhaler according to claim 23, wherein the locking mechanism is configured to be mechanical and/or electronic and/or electromechanical and/or electromagnetic and/or computer-implemented or a combination of the aforementioned.

25. The inhaler according to claim 23, further comprising an element configured and adapted to convert the data signal formed by the at least one sensor into a biometric signature, wherein the biometric signature forms an electronic lock as the locking mechanism for the actuating mechanism.

26. The inhaler according to claim 22, wherein the at least one sensor is an optical, capacitive, optical-capacitive or ultrasound-based skin print sensor or comprises such a sensor.

27. The inhaler according to claim 25, wherein the electronic control unit is configured as a programmable logic control unit which is configured and adapted to store the biometric signature and to read out the biometric signature from the storage device.

28. The inhaler according to claim 27, further comprising an element configured and adapted for digital verification of the identity and/or age of the person using the inhaler.

29. The inhaler according to claim 28, wherein for initial verification, the programmable logic control unit is configured and adapted to form a data set formed from the biometric signature and a digital proof of identity and/or age of the user and to store and read out the data set.

30. The inhaler according to claim 29, wherein for re-verification, the programmable logic control unit is configured and adapted to compare the stored data set with a data signal currently formed from the characteristics during each individual use.

31. The inhaler according to claim 22, further comprising an element configured and adapted to establish a wireless connection to a network-enabled terminal device.

32. The inhaler according to claim 22, further comprising:
a vaporiser cartridge associated with the cartridge carrier;
an energy source associated with a unit formed from the cartridge carrier and vaporiser cartridge;
wherein;
the vaporiser cartridge has a hollow body with a continuous flow channel;
the storage tank is configured to receive the liquid inhalation medium and has at least one access opening to the flow channel; and
a vaporiser unit extends over the entire at least one access opening and is arranged in a region of each of the at least one access openings.

33. The inhaler according to claim 32, wherein the vaporiser unit has a wick member and a heating member, the vaporiser unit comprising electrical contacts for electrically contacting the energy source.

34. The inhaler according to claim 32, wherein the vaporiser unit with the energy source forms the actuating mechanism, wherein the actuating mechanism is controlled and/or regulated by the electronic control unit.

35. A system, configured and adapted for inhaling inhalation medium enriched with active ingredients and/or flavouring substances, comprising:
an inhaler according to claim 22; and
a network-enabled terminal;
wherein the inhaler and the terminal device are wirelessly in signal connection with each other.

36. The system according to claim 35, wherein the inhaler and/or the terminal device are wirelessly connected to a network.

37. The system according to claim 35, wherein the terminal has a user-defined account which is linked to the inhaler.

38. A method for inhaling inhalation medium enriched with active ingredients and/or flavouring substances by an inhaler having a mouthpiece, comprising steps of:
sucking at the mouthpiece of the inhaler by a user; and
simultaneously actuating an actuating mechanism of the inhaler, whereby the inhaler is activated for inhaling the inhalation medium;
establishing a connection between an identity and/or age of the user and characteristics of lips of the user and storing the characteristics before sucking for the first time; and
during sucking comparing the characteristics of the lips of a person currently using the inhaler with the stored characteristics, in such a manner that activation of the actuating mechanism only takes place if the respective characteristics match to a defined minimum degree.

39. The method according to claim 38, wherein an initially detected and saved connection is stored as authorisation to use the inhaler.

40. The method according to claim 38, wherein a lip wrinkle pattern of the user is detected by means of a sensor and converted into a biometric signature, wherein the biometric signature is linked to an authorisation to use the inhaler and deactivates the actuating mechanism as an electronic lock until the authorised person sucks on the inhaler.

41. The method according to claim 38, further comprising performing the method with a system comprising:
an inhaler comprising;
a cartridge carrier;
a storage tank which contains the inhalation medium;
a mouthpiece associated with the cartridge carrier;
an actuating mechanism for releasing the inhalation medium out of the storage tank in a direction of the mouthpiece;
at least one sensor associated with the mouthpiece, the sensor being configured and adapted to detect characteristics of lips of a person using the inhaler and to provide a data signal formed from the characteristics; and
an electronic control unit, the sensor and the actuating mechanism being connected to the electronic control unit such that the inhalation medium is released on a basis of the data signal; and
a network-enabled terminal;
wherein the inhaler and the terminal device are wirelessly in signal connection with each other.

42. The method according to claim 38, wherein the step of comparing during sucking comprises comparing during each suck.

43. A method for controlling an inhaler having a mouthpiece with a sensor, as a result of which the inhaler is activated for inhaling an inhalation medium, comprising steps of:
recording of data;
verifying the recorded data of the current user with initially stored data; and
if the initially stored data matches the recorded data of the current user to a defined minimum degree, enabling the actuating mechanism of the inhaler.

44. The method according to claim 43, wherein the inhaler having a mouthpiece with a sensor comprises
a cartridge carrier;
a storage tank which contains the inhalation medium;
a mouthpiece associated with the cartridge carrier;
an actuating mechanism for releasing the inhalation medium out of the storage tank in a direction of the mouthpiece;
at least one sensor associated with the mouthpiece, the sensor being configured and adapted to detect characteristics of lips of a person using the inhaler and to provide a data signal formed from the characteristics; and
an electronic control unit, the sensor and the actuating mechanism being connected to the electronic control unit such that the inhalation medium is released on a basis of the data signal.

45. The method according to claim 43, wherein the step of recording of data comprises recording of data comprising characteristics of lips of a person currently using the inhaler by the sensor.

46. The method according to claim 43, wherein the step of verifying the recorded data of the current user with initially stored data comprises verifying the recorded data of the current user with initially stored characteristics of the lips.

47. The method according to claim 43, further comprising, if the initially stored data matches the recorded data of the current user to a defined minimum degree, activating the actuating mechanism of the inhaler.
